(19) United States
(12) Patent Application Publication
(10) Pub. No.: US 2009/0093627 A1
(43) Pub. Date: Apr. 9, 2009

(54) Process for preparing intermediates of ezetimibe by microbial reduction

(76) Inventors: Lorand Szabo, Godollo (HU); Laszlo Toth, Balmazujvaros (HU); Nurit Perlman, Kfar Saba (IL)

Correspondence Address: KENYON & KENYON LLP, ONE BROADWAY, NEW YORK, NY, 10004, US

(21) Appl. No.: 12/231,438

(22) Filed: Sep. 2, 2008

Related U.S. Application Data
(60) Provisional application No. 60/967,058, filed on Aug. 30, 2007.
(60) Provisional application No. 61/073,343, filed on Jun. 17, 2008.

(51) Int. Cl. C07D 205/08; C12P 17/10

(52) U.S. Cl. 540/200; 435/71.1

(57) ABSTRACT

Processes of preparing an ezetimibe intermediate by microbial reduction and further converting the intermediate to ezetimibe are provided. Also provided is an ezetimibe intermediate with high diastereomeric excess.

### CROSS-REFERENCE TO RELATED APPLICATIONS

This application claims the benefit of Provisional Application Ser. No. 60/967,058, filed Aug. 30, 2007, and Provisional Application Ser. No. 61/073,343, filed Jun. 17, 2008, each of which is incorporated herein by reference in its entirety.

### FIELD OF THE INVENTION

The invention relates to microbial reduction processes of an ezetimibe intermediate to obtain ezetimibe or a derivative thereof.

### BACKGROUND OF THE INVENTION

Hydroxy-alkyl substituted azetidinones are useful as hypercholesterolemia agents in the treatment and prevention of atherosclerosis. Ezetimibe, 1-(4-fluorophenyl)-3(R)-[3-(4-fluorophenyl)-3(S)-hydroxypropyl]-4(S)-(4-hydroxyphenyl)-2-azetidinone, is a selective inhibitor of intestinal cholesterol and related phytosterol absorption. The empirical formula for ezetimibe is C₂₄H₂₁F₂NO₃, and its molecular weight is 409.4.

Ezetimibe is a white, crystalline powder that is freely to very soluble in ethanol, methanol, and acetone and practically insoluble in water. Ezetimibe has the following chemical structure:

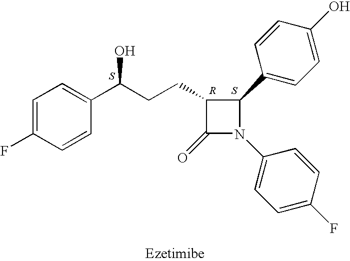

Ezetimibe is the active ingredient in the drug sold under the brand name ZETIA®, which is manufactured by Merck/Schering-Plough Pharmaceuticals. ZETIA® has been approved by the United States Food and Drug Administration for use in patients with high cholesterol to reduce low density lipoprotein (“LDL”) cholesterol and total cholesterol. ZETIA® is available as a tablet for oral administration.

Ezetimibe can be prepared by reducing (3R,4S)-4-((4-benzyloxy)phenyl)-1-(4-fluorophenyl)-3-(3-(4-fluorophenyl)-3-oxopropyl)-2-azetidinone (“Compound 1” or “BZT-ketone”) with borane dimethyl sulfide complex or borane tetrahydrofuran complex in tetrahydrofuran in the presence of Corey's reagent and subsequently deprotecting the benzyl group, as shown in Scheme 1 below. The process is disclosed in U.S. Pat. Nos. 5,631,365 (“the '365 patent”) and 6,627,757, each of which is incorporated herein by reference in its entirety. The starting material, Compound 1 or a similar compound, can be prepared by processes known in the art, for example, those disclosed in the '365 patent.

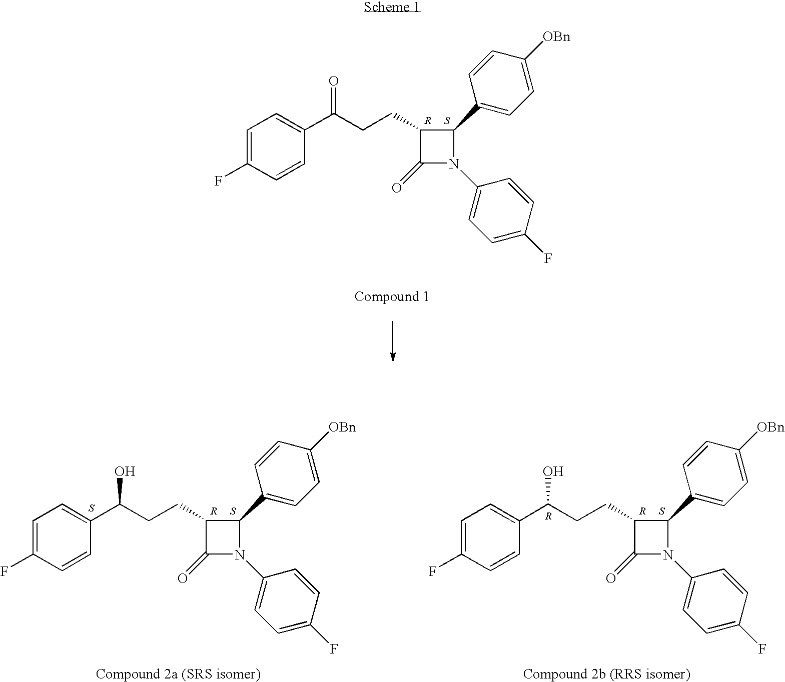

The reduction process produces two isomers, (3R,4S)-4-((4-benzyloxy)phenyl)-1-(4-fluorophenyl)-3-((S)-3-(4-fluorophenyl)-3-hydroxypropyl)-2-azetidinone (“Compound 2a” or “BZT”) and (3R,4S)-4-((4-benzyloxy)phenyl)-1-(4-fluorophenyl)-3-((R)-3-(4-fluorophenyl)-3-hydroxypropyl)-2-azetidinone (“Compound 2b” or “BZT RRS isomer”). Compound 2a is the desired isomer that produces ezetimibe of the proper chirality. Compound 2b is an undesirable isomer that is very difficult to remove during both the reduction as well as the final synthesis to form ezetimibe. It has been reported that Compound 2b is typically produced in about 8 to 10% yield during the reduction process.

The '365 patent refers to the reduction of BZT-ketone to BZT by (R)-(+)-2-methyl-CBS-oxazaborolidine (“CBS”) and borohydride dimethylsulfide complex (“BMS”), as illustrated below.

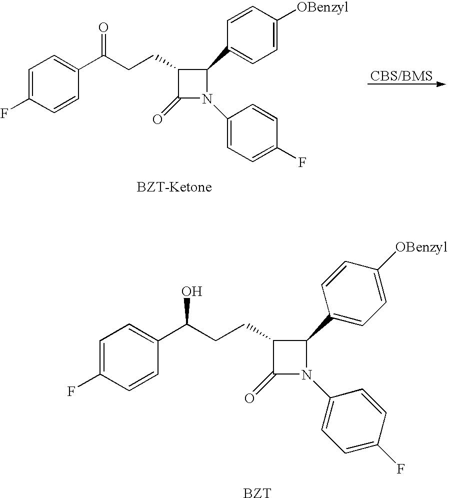

U.S. Pat. No. 6,133,001 refers to a process for stereoselective microbial reduction of ezetimibe-ketone to ezetimibe, as illustrated below.

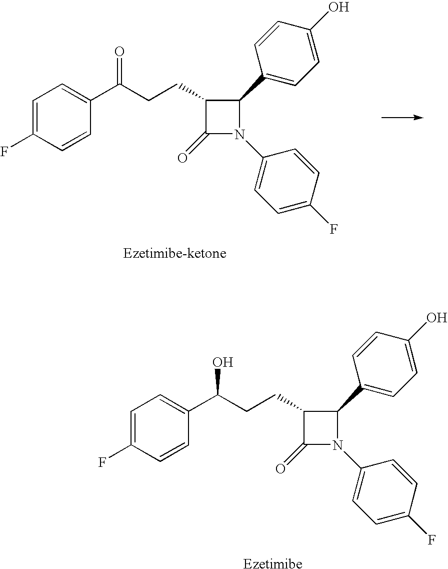

PCT publication no. WO 2005/066120 refers to a stereoselective reduction of ezetimibe-ketone to ezetimibe with (−)-B-chlorodiisopinocampheylborane (“DIP-Cl”).

PCT publication no. WO 2007/030721 (“the '721 publication”), which is incorporated herein by reference in its entirety, refers to reduction processes of protected or unprotected ezetimibe-ketone to the corresponding alcohol using chiral catalysts or hydrogenation.

U.S. application Ser. No. 12/135,847, which is incorporated herein by reference in its entirety, refers to a reduction process of protected or unprotected ezetimibe-ketone to the corresponding alcohol using an isolated, synthesized, or purified ketoreductase.

Bertrand et al., Process for Preparing Ezetimibe Intermediate by Enantioselective CBS Catalyzed Ketone Reduction with BH₃-DEA Prepared in situ, Tetrahedron letters, 48, 2123-2125 (2007), refers to a reduction process using CBS and BH₃-diethylaniline.

There is a need for additional and improved methods for preparing ezetimibe intermediates.

### SUMMARY OF THE INVENTION

In one embodiment, the present invention encompasses a process comprising combining (3R,4S)-4-((4-benzyloxy)phenyl)-1-(4-fluorophenyl)-3-(3-(4-fluorophenyl)-3-oxopropyl)-2-azetidinone and a Rhodococcus fascians strain, whereby (3R,4S)-4-((4-benzyloxy)phenyl)-1-(4-fluorophenyl)-3-((S)-3-(4-fluorophenyl)-3-hydroxypropyl)-2-azetidinone is obtained.

In one embodiment, the invention encompasses a process comprising preparing (3R,4S)-4-((4-benzyloxy)phenyl)-1-(4-fluorophenyl)-3-((S)-3-(4-fluorophenyl)-3-hydroxypropyl)-2-azetidinone according to the above process, and further converting it to ezetimibe.

In one embodiment, the invention encompasses (3R,4S)-4-((4-benzyloxy)phenyl)-1-(4-fluorophenyl)-3-((S)-3-(4-fluorophenyl)-3-hydroxypropyl)-2-azetidinone having a diastereomeric excess of about 99% or more.

### DETAILED DESCRIPTION OF THE INVENTION

The present invention provides a new process for preparing BZT from BZT-ketone by microbial reduction. Preferably, this process has very high stereoselectivity.

As used herein, the term “d.e.” refers to diastereomeric excess, defined as: (mole fraction of BZT) minus (mole fraction of BZT RRS isomer).

$$d . e . = \frac{( \% BZT - \% BZT RRS isomer )}{( \% BZT + \% BZT RRS isomer )}$$

As used herein, the term “room temperature” refers the ambient temperature of about 15° C. to about 30° C.

As used herein, the term “vacuum” refers to a pressure of about to 2 mmHg to about 100 mmHg.

As used herein, the term “BZT” refers to (3R,4S)-4-((4-benzyloxy)phenyl)-1-(4-fluorophenyl)-3-((S)-3-(4-fluorophenyl)-3-hydroxypropyl)-2-azetidinone having the following chemical structure (III):

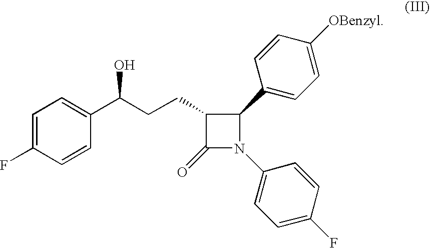

As used herein, the term “BZT-ketone” refers to (3R,4S)-4-((4-benzyloxy)phenyl)-1-(4-fluorophenyl)-3-(3-(4-fluorophenyl)-3-oxopropyl)-2-azetidinone having the following chemical structure (IV):

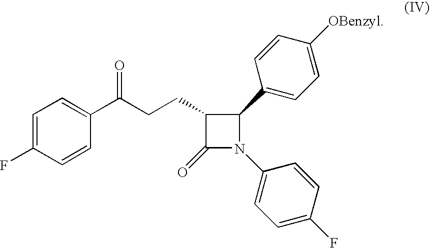

The present invention encompasses a process comprising combining (3R,4S)-4-((4-benzyloxy)phenyl)-1-(4-fluorophenyl)-3-(3-(4-fluorophenyl)-3-oxopropyl)-2-azetidinone with a Rhodococcus fascians strain, whereby (3R,4S)-4-((4-benzyloxy)phenyl)-1-(4-fluorophenyl)-3-((S)-3-(4-fluorophenyl)-3-hydroxypropyl)-2-azetidinone is obtained.

While many microorganisms have reduction capabilities, it cannot be predicted which microorganism can reduce which substrate. A microorganism's ability to reduce a substrate (e.g. a ketone) depends on the structure of the substrate as well as the structure of the active site of the enzyme within the cells of the microorganism.

Optionally, the Rhodococcus fascians strain used in the processes of the present invention is obtained from any one of the following resources: American Type Culture Collection (ATCC), including, for example, Cat Nos. ATCC 12975, ATCC 13000, ATCC 21057, ATCC 21950, ATCC 35014, and ATCC 12974; Institute for Fermentation Osaka (IFO); National Institute of Technology and Evaluation (“NITE”) Biological Resource Center (“NBRC,” which includes the biological resources transferred from IFO); German Resource Centre for Biological Material (Deutsche Sammlung von Mikroorganismen und Zell-Kulturen (“DSMZ”), including, for example, Cat No. DSM 20669; and Agricultural Research Service (“ARS”) Culture Collection, National Center for Agricultural Utilization Research (“NCAUR,” formerly Northern Regional Research Laboratory (“NRRL”)).

Preferably, the Rhodococcus fascians strain is ATCC No. 12974. The Rhodococcus fascians strain ATCC No. 12974 is also available from the following sources: The French collection of plant pathogenic bacterial (Collection Francaise de Bacteries Phytopathogenes, “CFBP”), CFBP No. 2401; Institut Pasteur Collection (Collection de l'Institute Pasteur, “CIP”), CIP No. 104713; International Collection of Micro-organisms from Plants (“ICMP”) ICMP No. 5833; IFO (now NBRC) No. 12155; Japan Collection of Microorganisms (“JCM”), JCM No. 10002; Belgian Co-ordinatd Collections of Micro-organisms (“BCCM™”)/Laboratory of Microbiology, Ghent University (“LMG”), LMG No. 3623; National Collection of Plant Pathogenic Bacteria (“NCPPB”), NCPPB No. 3067; NRRL No. B-16937; and All Russian Collection of Microorganisms (“VKM”), VKM No. Ac-1462.

Preferably, prior to the combination, the Rhodococcus fascians strain is proliferated in a medium. Any suitable solid or liquid medium for culturing microorganisms known in the art can be used. Optionally, the medium comprises calf brains (preferably about 7.7 g/l of medium), beef heart (preferably about 9.8 g/l of medium), proteose peptone (preferably about 10.0 g/l of medium), dextrose (preferably about 2.0 g/l of medium), sodium chloride (preferably about 5.0 g/l of medium), disodium phosphate (preferably about 2.5 g/l of medium), and optionally agar (preferably about 15 g/l of medium). Preferably, the medium is equivalent to the medium commercially available under the brand name Dilfcoo Brain Heart Infusion Agar, available through Becton, Dickinson and Company as BD Catalog No. 241830, which comprises about 7.7 g/l of calf brains, about 9.8 g/l of beef heart, about 10.0 g/l of proteose peptone, about 5.0 g/l of sodium chloride, about 2.5 g/l of disodium phosphate, and about 15 g/l of agar. Optionally, the medium a YPD medium comprising yeast extract (preferably about 10.0 g/l of medium), peptone (preferably about 20.0 g/l of medium), dextrose (preferably about 20.0 g/l of medium), and optionally agar (preferably about 15 g/l of medium). Preferably, the YPD medium is equivalent to the medium commercially available under the brand name Difco® YPD broth, as BD Catalog No. 242810, which comprises about 10.0 g/l of yeast extract, about 20.0 g/l of peptone, and about 20.0 g/l of dextrose. Preferably, the bacteria are proliferated for about 1 to about 6 days, preferably for about 4 days, on a solid medium, preferably on a medium comprising calf brains, beef heart, proteose peptone, dextrose, sodium chloride, disodium phosphate, and agar.

Preferably, after the proliferation step, the proliferated bacteria are inoculated into a liquid medium, which is preferably a YPD medium comprising yeast extract, peptone, and dextrose, to obtain a fermentation broth. Preferably, the fermentation broth is incubated for about 12 hours to about 3 days, preferably about 1 day. Preferably, the fermentation broth is incubated at about 200 to about 400 rotations per minute (“rpm”), preferably about 300 rpm. Preferably, the fermentation broth is incubated at a temperature of about 20° C. to about 40° C., preferably about 28° C. Preferably, after the above incubation step, at least part of the fermentation broth is transferred into fresh liquid medium, preferably YPD medium, and further incubated for about 1 day to about 3 days, preferably about 2 days, at about at about 200 to about 400, preferably about 300 rpm, and at a temperature of about 20° C. to about 40° C., preferably about 28° C.

Preferably, the process comprises combining an organic solvent with the (3R,4S)-4-((4-benzyloxy)phenyl)-1-(4-fluorophenyl)-3-(3-(4-fluorophenyl)-3-oxopropyl)-2-azetidinone and the Rhodococcus fascians strain. Preferably, the BZT-ketone is dissolved in the organic solvent. Preferably, the organic solvent is selected from a group consisting of dimethyl sulfoxide (“DMSO”), alcohol, and mixtures thereof. Preferably, the alcohol is an aliphatic alcohol, preferably a C₁₋₄ aliphatic alcohol. Preferably, the organic solvent is a mixture of DMSO and ethanol. More preferably, the organic solvent is a mixture of about 50% ethanol and about 50% DMSO by volume.

Preferably, the solution of BZT-ketone is fed into the fermentation broth, preferably about 1 to about 2 days after the start of the incubation. Preferably, the initial concentration of BZT-ketone in the fermentation broth is about 0.5 g/l to about 10 g/L, about 1 g/l or more, or about 2 g/l or more. Preferably, the obtained fermentation broth is further incubated for about 2 days to about 8 days, preferably for about 4 days.

Preferably, after the feeding step or the incubation step, the fermentation broth is extracted with an organic solvent. The extracting organic solvent may be any water immiscible solvent in which the BZT is soluble. Preferably, the organic solvent is selected from dichloromethane (“DCM”), ethyl acetate, and mixtures thereof. More preferably, the organic solvent is dichloromethane. Preferably, the volume ratio between the organic solvent and the fermentation broth is between about 0.5:1 and about 2:1, preferably between about 1:1 and about 1.5:1, preferably about 1.25:1.

Preferably, the obtained extract is further concentrated. Preferably, the concentration is performed under vacuum. Preferably, after the concentration step, the extract is further dissolved in an organic solvent. Preferably, the organic solvent is selected from a group consisting of ethyl acetate, DCM, butyl acetate, and mixtures thereof. Preferably, the organic solvent is ethyl acetate.

Optionally, the BZT obtained is recovered. Preferably, the BZT is recovered from the solution by crystallization or by removing the solvents by evaporation or distillation. Optionally, the BZT obtained is purified, preferably by crystallization.

In one embodiment, the invention encompasses a process comprising combining (3R,4S)-4-((4-benzyloxy)phenyl)-1-(4-fluorophenyl)-3-(3-(4-fluorophenyl)-3-oxopropyl)-2-azetidinone with a Rhodococcus fascians strain, and further converting the (3R,4S)-4-((4-benzyloxy)phenyl)-1-(4-fluorophenyl)-3-((S)-3-(4-fluorophenyl)-3-hydroxypropyl)-2-azetidinone obtained to ezetimibe. The conversion may be done according to known methods. For example, the conversion may be done by hydrogenation with a palladium on carbon catalyst, as described in Example 10 of the '721 publication and Example 6 of the '365 patent, or by transfer hydrogenation with ammonium formate and acetic acid with a palladium on carbon catalyst, as described in Wu et al., A Novel One-Step Diastereo- and Enantioselective Formation of trans-Azetidinones and Its Application to the Total Synthesis of Cholesterol Absorption Inhibitors, J. Org. Chem., Vol. 64 (10): 3714-3718 (1999).

In one embodiment, the invention encompasses (3R,4S)-4-((4-benzyloxy)phenyl)-1-(4-fluorophenyl)-3-((S)-3-(4-fluorophenyl)-3-hydroxypropyl)-2-azetidinone having a d.e. of about 99% or more, preferably about 99.5% or more, and more preferably about 99.8% or more.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

### General

pH Measurements:

The pH values were measured using a potentiometric electrode at room temperature.

Thin Layer Chromatography (“TLC”):

10 μl of sample was run on TLC Silica gel 60 F254, aluminum sheet 10×20, MERCK, Cat. No. 5554, in n-hexane/ethyl acetate=70:30 v/v.

Determination of RRS-Isomer in BZT by HPLC:

| Column & Packing: | Daicel ®, Chiralpak ® AD-H 150 × 4.6 mm, | |
|---|---|---|
| | 5 μm | |
| Eluent: | Heptane: Isopropyl alcohol (“IPA”): | |
| | trifluoroacetic acid (“TFA”) (80:20:0.1) | |
| Run time | 40 | min |
| Sample volume: | 10 | μL |
| Flow Rate: | 1.0 | ml/min |
| Detector: | 248 | nm |
| Column temperature: | 35° | C. |
| Autosampler temperature | 10° | C. |

| | |
|---|---|
| Diluent | Ethanol |

The % BZT and % BZT RRS isomer were determined by the area under the corresponding HPLC peaks.

System Suitability Solution:

1 mg/ml solution of BZT System Suitability Marker (BZT and BZT RRS isomer) was made in a volumetric flask.

System Suitability Test (“SST”):

Inject the System Suitability Solution into the column.

The resolution between BZT and BZT RRS isomer peak in System Suitability Solution was not less than 2.8.

Typical Retention Times:

| | Retention | Relative |
|---|---|---|
| Compound | Time (min) | Retention Time |
| BZT | 17 | 1 |
| BZT RRS isomer | 21.5 | 1.26 |
| RRT 0.79 isomer (which has same | 13.7 | 0.79 |
| molecular weight as BZT) | | |

### Example 1

### Microbial Reduction of BZT-Ketone to BZT Using Rhodococcus fascians

Rhodococcus fascians (Strain ATCC No. 12974) was proliferated for 4 days on Difco® Brain Heart Infusion Agar (BD Cat No. 241830). One loop of mycelia was inoculated into 25 ml of Yeast-Peptone-Dextrose media (1% yeast extract, 2% bacto-peptone, 2% glucose) at a pH of 5.5 in 100 ml flask, and incubated for 1 day at 300 rpm and 28° C. 800 μm of the inoculum was transferred into 20 ml of Yeast-Peptone-Dextrose media in a 100 ml flask, and incubated for 48 hours at 300 rpm and 28° C. 800 μl of 25 mg/ml BZT-ketone dissolved in a 50%/50% v/v ethanol/DMSO mixture was fed into the fermentation broth (final concentration of BZT-ketone in broth: 1 mg/ml) and further incubated for 96 hours. 800 μl of the fermentation broth was extracted with 600 μl dichloromethane. 350 μl of the extract was concentrated under vacuum and dissolved in 50 μl of ethyl acetate. 10 μl of the solution was run on TLC and also measured by HPLC. Based on the area under the HPLC peaks, at least 10% of the fed BZT-ketone was converted to BZT with 99.5% d.e.

### Comparative Example 1

### Microbial Reduction of BZT-Ketone Using Geotrichum candidum

The procedure of Example 1 was followed, except that the Rhodococcus fascians was replaced with Geotrichum candidum (Strain ATCC No. 12252) and the proliferation medium was replaced with a SIM6 medium comprising 3.5% soy meal, 5% dextrin, 0.5% glucose, 0.5% CaCO₃, and 2 mg/l CoCl₂ (pH=6.0). The TLC results showed no BZT-ketone conversion to BZT.

### Comparative Example 2

### Microbial Reduction of BZT-Ketone Using Other Microorganisms

The procedure of Example 1 was followed, with the Rhodococcus fascians being replaced by Zygosaccharomyces rouxii, Sacharomyces bayanus, Saccharomyces uvarum, and Saccharomyces cerevisiae, respectively. The TLC results showed no BZT-ketone conversion to BZT.

1. A process comprising combining (3R,4S)-4-((4-benzyloxy)phenyl)-1-(4-fluorophenyl)-3-(3-(4-fluorophenyl)-3-oxopropyl)-2-azetidinone with a Rhodococcus fascians strain, whereby (3R,4S)-4-((4-benzyloxy)phenyl)-1-(4-fluorophenyl)-3-((S)-3-(4-fluorophenyl)-3-hydroxypropyl)-2-azetidinone is obtained.

2. The process of claim 1, further comprising combining an organic solvent with the (3R,4S)-4-((4-benzyloxy)phenyl)-1-(4-fluorophenyl)-3-(3-(4-fluorophenyl)-3-oxopropyl)-2-azetidinone and the Rhodococcus fascians strain.

3. The process of claim 2, wherein the (3R,4S)-4-((4-benzyloxy)phenyl)-1-(4-fluorophenyl)-3-(3-(4-fluorophenyl)-3-oxopropyl)-2-azetidinone is dissolved in the organic solvent prior to combining with the Rhodococcus fascians strain.

4. The process of claim 2, wherein the organic solvent is selected from the group consisting of dimethyl sulfoxide, alcohol, and mixtures thereof.

5. The process of claim 4, wherein the alcohol is an aliphatic alcohol.

6. The process of claim 5, wherein the organic solvent is a mixture of DMSO and ethanol.

7. The process of claim 6, wherein the organic solvent is a mixture of about 50% ethanol and about 50% DMSO by volume.

8. The process of claim 1, wherein prior to the combining, the Rhodococcus fascians strain is proliferated on a medium comprising calf brains, beef heart, proteose peptone, dextrose, sodium chloride, disodium phosphate, and agar.

9. The process of claim 1, wherein prior to the combining, the Rhodococcus fascians strain is proliferated on a medium comprising yeast extract, peptone, and dextrose.

10. The process of claim 1, wherein the Rhodococcus fascians strain undergoes incubation in a fermentation broth.

11. The process of claim 10, wherein the incubation is after the proliferation.

12. The process of claim 10, wherein the (3R,4S)-4-((4-benzyloxy)phenyl)-1-(4-fluorophenyl)-3-(3-(4-fluorophenyl)-3-oxopropyl)-2-azetidinone is combined with the Rhodococcus fascians strain about 1 to about 2 days after the start of the incubation.

13. The process of claim 10, wherein the mixture of (3R,4S)-4-((4-benzyloxy)phenyl)-1-(4-fluorophenyl)-3-(3-(4-fluorophenyl)-3-oxopropyl)-2-azetidinone and the Rhodococcus fascians strain is incubated for about 2 to about 8 days.

14. The process of claim 13, wherein the mixture of (3R,4S)-4-((4-benzyloxy)phenyl)-1-(4-fluorophenyl)-3-(3-(4-fluorophenyl)-3-oxopropyl)-2-azetidinone and the Rhodococcus fascians strain is incubated for about 4 days.

15. The process of claim 1, wherein the (3R,4S)-4-((4-benzyloxy)phenyl)-1-(4-fluorophenyl)-3-((S)-3-(4-fluorophenyl)-3-hydroxypropyl)-2-azetidinone is extracted with an extracting organic solvent.

16. The process of claim 15, wherein the extracting organic solvent is selected from dichloromethane, ethyl acetate, and mixtures thereof.

17. The process of claim 16, wherein the extracting organic solvent is dichloromethane.

18. The process of claim 15, wherein the obtained extract is concentrated.

19. The process of claim 1, wherein the (3R,4S)-4-((4-benzyloxy)phenyl)-1-(4-fluorophenyl)-3-((S)-3-(4-fluorophenyl)-3-hydroxypropyl)-2-azetidinone is recovered.

20. The process of claim 1, further comprising converting the (3R,4S)-4-((4-benzyloxy)phenyl)-1-(4-fluorophenyl)-3-((S)-3-(4-fluorophenyl)-3-hydroxypropyl)-2-azetidinone to 1-(4-fluorophenyl)-3(R)-[3-(4-fluorophenyl)-3(S)-hydroxypropyl]-4(S)-(4-hydroxyphenyl)-2-azetidinone.

21. (3R,4S)-4-((4-benzyloxy)phenyl)-1-(4-fluorophenyl)-3-((S)-3-(4-fluorophenyl)-3-hydroxypropyl)-2-azetidinone having a diastereomeric excess of about 99% or more.

22. The (3R,4S)-4-((4-benzyloxy)phenyl)-1-(4-fluorophenyl)-3-((S)-3-(4-fluorophenyl)-3-hydroxypropyl)-2-azetidinone of claim 21 having a diastereomeric excess of about 99.5% or more.

23. The (3R,4S)-4-((4-benzyloxy)phenyl)-1-(4-fluorophenyl)-3-((S)-3-(4-fluorophenyl)-3-hydroxypropyl)-2-azetidinone of claim 21 having a diastereomeric excess of about 99.8% or more.
